SEQUENCE LISTING (52 sequences)

SEQ ID NO: 1 - DNA, 263 bp, Artificial Sequence
  Synthesized
atggaactgaagcatagcattagtgattatacagaagctgaatttttacaacttgtaacaacaatttgtaatgcggacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggctaaaa

SEQ ID NO: 2 - DNA, 255 bp, Artificial Sequence
  Synthesized
atgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattgg

SEQ ID NO: 3 - protein, 11 aa, Escherichia coli
IPTTENLYFQG

SEQ ID NO: 4 - DNA, 410 bp, Artificial Sequence
  Synthesized
tagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 5 - DNA, 354 bp, Artificial Sequence
  Synthesized
aaagaagataagaaaaggagtgctgaaaataatttaaacgatgaaaagaataagcccagaaaaggttttaaagattacgggcatgattatcatccagctccgaaaactgagaatattaaagggcttggtgatcttaagcctgggataccaaaaacaccaaagcagaatggtggtggaaaacgcaagcgctggactggagataaagggcgtaagatttatgagtgggattctcagcatggtgagcttgaggggtatcgtgccagtgatggtcagcatcttggctcatttgaccctaaaacaggcaatcagttgaaaggtccagatccgaaacgaaatatcaagaaatatctttga

SEQ ID NO: 6 - protein, 97 aa, Enterobacter sp.
MFKEKLEDYTEEEFLNFLGGLRSTMKDGKPLKGKELEMYWDSLVDHFIEITQHPSGSDLIFYPKSQGDDKPENILKIVKEWRRSQGLPLFKDSKQSR

SEQ ID NO: 7 - protein, 96 aa, Enterobacter sp.
MYNFKDKIEDYTEREFIELLGEFTNPTGDNAQLRGEELDKYWDDLEEHLTRITQHPLMSDLIYYPAKKGDDKPENILKIVKEWRRSQGLPLFKDSE

SEQ ID NO: 8 - protein, 88 aa, Enterobacter sp.
MKLKENISDYTESEFIDFLRVIFSENESDTDETLDPLLEYFEKITEYPGGTDLIYYPETESDGTPEGILNIIKEWRESQGLPCFKKSK

SEQ ID NO: 9 - protein, 92 aa, Enterobacter sp.
MELKNNLEDYTEDEFIEFLNNFFEPPEELTGDELSKFIDNLLRHFNKITQHPDGGDLIFYPSEEREDSPEGVIEELKRWRKSQRLPCFKENK

SEQ ID NO: 10 - protein, 90 aa, Enterobacter sp.
MIFKEKLEDYTEEEFLEFLRGLSSQHIQLHGDEFVKHMDRLVKHFVKVTEHPAQTDVIFYPEEGQEDTPEGILKTIKEWRAENGKPGFKR

SEQ ID NO: 11 - protein, 90 aa, Enterobacter sp.
MDIKNNLSDYTESEFLEIIEEFFKNKSGLKGSELEKRMDKLVKHFEEVTSHPRKSGVIFHPKPGFETPEGIVKEVKEWRAANGLPGFKAG

SEQ ID NO: 12 - protein, 86 aa, Enterobacter sp. [sequence omitted]

SEQ ID NO: 13 - protein, 86 aa, Enterobacter sp.
MELKHSISDYTEAEFLEFVKKICRAEGATEEDDNKLVREFERLTEHPDGSDLIYYPRDDREDSPEGIVKEIKEWRAANGKSGFKQG

SEQ ID NO: 14 - protein, 87 aa, Enterobacter sp.
MELKNSISDYTEAEFVQLLKEIEKENVAATDDVLDVLLEHFVKITEHPDGTDLIYYPSDNRDDSPEGIVKEIKEWRAANGKPGFKQG

SEQ ID NO: 15 - protein, 85 aa, Enterobacter sp.
MELKNSISDYTETEFKKIIEDIINCEGDEKKQDDNLEHFISVTEHPSGSDLIYYPEGNNDGSPEAVIKEIKEWRAANGKSGFKQG

SEQ ID NO: 16 - protein, 85 aa, Enterobacter sp.
MKLNKKLEDYTEAEFLEFARKVCNADYATEDEANVAVQDFIRLSEHPDGTDILFYPSSGQDDSPEGIVKQIKEWRAKSGKPGFKK

SEQ ID NO: 17 - protein, 85 aa, Enterobacter sp.
MANKTLADYTEQEFIEFIEKIKKADFATESEHDEAIYEFSQLTEHPDGWDLIYHPQAGADNSPAGVVKTVKEWRAANGKPGFKKS

SEQ ID NO: 18 - protein, 87 aa, Enterobacter sp.
MKSKISEYTEKEFLEFVEDIYTNNKKKFPTEESHIQAVLEFKKLTEHPSGSDLLYYPNENREDSPAGVVKEVKEWRASKGLPGFKAG

SEQ ID NO: 19 - protein, 85 aa, Enterobacter sp.
MELKNKLEDYTEAEFLVLLNKIFDGECKTENEYHSLVKHIEIITDHPRRNGLIFYPENGVEDSPGGVLKVIKEWRAKNGKPGFKK

SEQ ID NO: 20 - protein, 83 aa, Enterobacter sp.
MENNVMNITENEFLSLIKKIFNGNFRTEEEESEAIDEFERISEHPSGGDLIFYPEDGIEDSPEGVLEVVKEWRTKNGKPGFKK

SEQ ID NO: 21 - protein, 85 aa, Enterobacter sp.
MELKEKYEDYTEHEFLEFIRNICEVNTDSQSLHSSWVRHFTKITEHPSGSDLIYYPEDGADDSPEGILELVKKWRAENGKPGFKK

SEQ ID NO: 22 - protein, 87 aa, Enterobacter sp.
MSEKTKLSDYTENEFLALIIEIHRANLEEPDHVLGGLLDHFSKITEHPSGYDLLYRPNPKENGKPEKVLEIVKQWRLANGKDGFKPS

SEQ ID NO: 23 - protein, 88 aa, Enterobacter sp.
MRDLKDKIYFYTEGEFLEMLEEIVNATSKDKSLKGKKLEKYLDTLVDHFIKITEHPKKGDLIFYPNSQEDGEPENRQRMAAFTGATLV

SEQ ID NO: 24 - protein, 85 aa, Enterobacter sp.
MGLKLDLTWFDKSTEDFKGEEYSKDFGDDGSVMESLGVPFKDNVNNGCFDVIAEWVPLLQPYFNHQIDISDNEYFVSFDYRDGDW

SEQ ID NO: 25 - protein, 85 aa, Enterobacter sp.
MGLKLHINWFDKRTEEFKGGEYSKDFGDDGSVIERLGMPFKDNINNGWFDVIAEWVPLLQPYFNHQIDISDNEYFVSFDYRDGDW

SEQ ID NO: 26 - protein, 85 aa, Enterobacter sp.
MGLKLHIHWFDKKTEEFKGGEYSKDFGDDGSVIESLGMPLKDNINNGWFDVEKPWVSILQPHFKNVIDISKFDYFVSFVYRDGNW

SEQ ID NO: 27 - protein, 85 aa, Enterobacter sp.
MGLKLNLTWFDKKTEEFKGEEYSKDFGDDGSVIESLGMPLKDNINNGCFDVKNEWVSLLQPYFKHKINLSDSSYFVSFDYRDGNW

SEQ ID NO: 28 - protein, 85 aa, Enterobacter sp.
MGLKLNLTWFDKKTEDFKGEEYSKDFGDDGSVIESLGMPLKDNINNGGFDVKKSWVPLLQPYFKNKIEVDKYWYQISFDYRDGNW

SEQ ID NO: 29 - protein, 84 aa, Enterobacter sp.
MGLKLNIAWFDKKTAEFIGEEYSGDLGDDGSVIEKLGLTIEDNINNGAFDVKKEWVPTLESCFKNKIETDKYWYKISFDYRDKW

SEQ ID NO: 30 - protein, 133 aa, Enterobacter sp.
ESKRNKPGKATGKGKPVGDKWLDDAGKDSGAPIPDRIADKLRDKEFKNFDDFRKKFWEEVSKDPDLSKQFKGSNKTNIQKGKAPFARKKDQVGGRERFELHHDKPISQDGGVYDMNNIRVTTPKRHIDIHRGK

SEQ ID NO: 31 - protein, 133 aa, Enterobacter sp.
ESKRNKPGKATGKGKPVGDKWLDDAGKDSGAPIPDRIADKLRDKEFKNFDDFRRKFWEEVSKDPELSKQFNPGNKKRLSQGLAPRARNKDTVGGRRSFELHHDKPISQDGGVYDMDNLRITTPKRHIDIHRGQ

SEQ ID NO: 32 - protein, 133 aa, Enterobacter sp.
ESKRNKPGKATGKGKPVGDKWLDDAGKDSGAPIPDRIADKLRDKEFKSFDDFRKAVWEEVSKDPELSKNLNPSNKSSVSKGYSPFTPKNQQVGGRKVYELHHDKPISQGGEVYDMDNIRVTTPKRHIDIHRGK

SEQ ID NO: 33 - protein, 133 aa, Enterobacter sp.
ESKRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIHRGK

SEQ ID NO: 34 - protein, 133 aa, Enterobacter sp.
KPARLTPGTVTGIGEDVSGIWLAGAGTGIGVPIPTRIANRLRGREFSSFDKFREAFWEEVANDPDLAEQFIQGNIERMRNGLSPKARRQDWAGDRTSYELHHVEHIANNGAVYDIDNLRVNTPKNHVENHRSG

SEQ ID NO: 35 - protein, 134 aa, Enterobacter sp.
KDPRSIPGVASGYGEPVTGVWLGDRTRAEGASIPTHIADQLRGRRFGDFASLRKATWIAVADDPELGKQSTQNNLEIMRGGGAPHPKLSDQAGGRTRFEIHHKNYISKGGAVYDIDNLVIMTSRQHIDHHRSQK

SEQ ID NO: 36 - protein, 133 aa, Enterobacter sp.
NTPRNQPGVVTGQGQRVEGNWLSLAGQDMGAPIPSQIADKLRGRRFNNFDDFRRAFWKEVGNDPELSQQFNSHNKEFLKKSYSPYSPRQEHIGNREKYEIHHVKFIKDGGEVYNLDNLRVTTPKRHIEIHSSK

SEQ ID NO: 37 - protein, 133 aa, Enterobacter sp.
ESPRDKPGVVTGKGEDIFGIWLADAGKDLGAPIPSQIADKLRGREFSSFDAFREGFWYAVGEDQTLINQFSRANQRLIKRGRSPFSLPSEQVGGRQRYELHHKEEIQYGGEVFHVDNLCVLTPKRHISIHKDQ

SEQ ID NO: 38 - protein, 134 aa, Enterobacter sp.
RDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRVADGGGVYNMGNLVAVTPKRHIEIHKGGK

SEQ ID NO: 39 - protein, 134 aa, Enterobacter sp.
RDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRIADGGGVYNMGNLVAVTPKRHIEIHKGGK

SEQ ID NO: 40 - protein, 134 aa, Enterobacter sp.
RDPRDEPGVATGNGQPVTGNWLAGASQGDGVPIPSQIADQLRGKEFKSWRDFREQFWMAVSKDPSALENLSPSNRYFVSQGLAPYAVPEEHLGSKEKFEIHHVVPLESGGALYNIDNLVIVTPKRHSEIHKELK

SEQ ID NO: 41 - protein, 133 aa, Enterobacter sp.
RSPRNMPGTASGKGQNVGNNWMGGTSTGDGAPVPSQIADKLRGKAFGSFDSFCRAFWKAVAADPDLSKQFYPDDIERMKLGRAPTVRFRDSVGKRVKVELHHKVEISKGGDVYNVDNLNALTPKRHIEIHKGN

SEQ ID NO: 42 - protein, 141 aa, Escherichia coli
EDPRKLPGVVTGRGVPLSPGTRWLDMSVSNNGNGAPIPAHIADKLRGREFKTFDEFREALWLEVSKDSVLLAQFIKSNQNNVSQGYSPYVPEEGYYYGPNEIVKKFQIHHVVAIEHGGGVYDIDNFRIVTPRLHDEIHYRR

SEQ ID NO: 43 - protein, 136 aa, Enterobacter sp.
KSPRYEAGTSTGHGAQVSDTWRKEAASLEGAPIPAQIAELLKSREFRNFDAFRRQFWKAVANDPELSKQFDEMSLSRMRKNGYSPIVDFPDSHLSQKTFILHHVIPISEGGGVYDMDNIRIVTPLSHNSIHYGTKP

SEQ ID NO: 44 - protein, 96 aa, Enterobacter sp.
GTKDYGHDYFPDPKTEDIKGLGELKEGKPKTPKQGGGGKRARWYGDKKRKIYEWDSQHGELEGYRASDGEHLGAFDPKTGKQVKGPDPKRNIKKYL

SEQ ID NO: 45 - protein, 96 aa, Enterobacter sp.
GVKDYGHDYHPDPKTEDIKGLGELKEGKPKTPKQGGGGKRARWYGDKGRKIYEWDSQHGELEGYRASDGQHLGSFEPKTGNQLKGPDPKRNIKKYL

SEQ ID NO: 46 - protein, 96 aa, Enterobacter sp.
GFKDYGHDYHPAPKTENIKGLGDLKPGIPKTPKQNGGGKRKRWTGDKGRKIYEWDSQHGELEGYRASDGQHLGSFDPKTGNQLKGPDPKRNIKKYL

SEQ ID NO: 47 - protein, 97 aa, Enterobacter sp.
GAKDYGHDYHPAPKTEDIKGLGDLKKGTPKTPMQGGGGRRKRWIGDKGRKIYEWDSQHGELEGYRASDGEHLGAFDPKTGKQIKGPDPKGRNIKKYL

SEQ ID NO: 48 - protein, 97 aa, Enterobacter sp.
GVKDYGHDYHPAPKTEEIKGLGELKKAPKKTPKQGGGGRRDRWIGDKGRKIYEWDSQHGELEGYRASDGEHIGAFDPKTGKQIKGPDPKGRNIKKYL

SEQ ID NO: 49 - protein, 96 aa, Enterobacter sp.
GLPQDGHDYHPAPKTEEITGVSGLRSAKKKTPKQSGGGKRDRWIDSKGRRIYEWDSQHGELEVYRVSDGEHLCSVDYKTGKELKPAVKGRNIKQYL

SEQ ID NO: 50 - protein, 92 aa, Enterobacter sp.
SYENGDHKYVPRPDVSDITGVDGLKRDRPKTPVQGGGGLRPRWKDEMGNIYEWDSYHGELEKYNKNGKHLGAFDYRTGQQIKPPEPGRKVEK

SEQ ID NO: 51 - protein, 92 aa, Enterobacter sp.
AKNPNDWKYIPAPRKDEITGITGLTEVKRKTSIQGGGKLRNRWKDREGYIYEWDSQHGTLEKYNHKGKHLGEFNYKTGKQTKSADPTRRIEP

SEQ ID NO: 52 - protein, 90 aa, Enterobacter sp.
VSKPGDHKYYDDPETLPAFPDTQRVKSKASVRGGGKKRARWLDGKGRIYEWDSKTGAIELYDKLGIHLGEFNHLTGERTKKAKPGRTTPK

(19) United States
(12) Patent Application Publication
(10) Pub. No.: US 2009/0233343 A1
(43) Pub. Date: Sep. 17, 2009

(54) AFFINITY PURIFICATION OF PROTEIN

(75) Inventors: Colin Kleanthous, York (GB); Theonie Georgiou, Cheshire (GB)

Correspondence Address: OCCHIUTI ROHLICEK & TSAO, LLP, 10 FAWCETT STREET, CAMBRIDGE, MA, 02138, US

(73) Assignee: University of York, York (GB)

(21) Appl. No.: 11/908,857

(22) PCT Filed: Mar. 13, 2006
(86) PCT No.: PCT/GB2006/000901

§ 371 Date: Mar. 27, 2008

(30) Foreign Application Priority Data
Mar. 17, 2005 (GB) 0505419.2

(51) Int. Cl. C12N 11/00; C12N 5/10; C12N 1/19; C12N 15/63; C07K 19/00; C07K 1/22; C07K 1/04; C07H 21/00

(52) U.S. Cl. 435/174; 435/325; 435/419; 435/243; 435/254.2; 435/320.1; 530/300; 530/344; 530/402; 536/23.2; 536/23.4

(57) ABSTRACT

We describe fusion proteins comprising a bacterial immunity polypeptide and their use in affinity purification of protein complexes.

The invention relates to a chimeric fusion protein for use in the affinity purification of polypeptides.

Affinity purification of biological molecules, for example proteins, is known in the art and allows the purification of molecules by exploiting the binding affinity of the target molecule for a molecular binding partner. For example, Staphylococcus Protein A will bind immunoglobulin G and this has been used to purify and/or concentrate antibodies from serum. Further examples of “affinity tags” include, maltose binding protein, glutathione S transferase, calmodulin binding protein and the engineering of polyhistidine tracks into proteins that are then purified by affinity purification on nickel containing matrices. In many cases commercially available vectors and/or kits can be used to fuse a protein of interest to a suitable affinity tag that is subsequently transfected into a host cell for expression and subsequent extraction and purification on an affinity matrix.

Genomic sequencing has resulted in a massive increase in the identification of genes encoding proteins. In some examples the function of a gene is not apparent simply by reference to its linear sequence and there is a desire to identify the function of these unassigned proteins and the protein partners with which they interact in a cell. A reverse genetic approach to the identification of gene function is a laborious and time consuming. A physical method that identifies the protein targets of proteins identified through genomic sequencing is attractive and may be a first step toward assigning a function to an unassigned open reading frame. In addition, it might also be desirable to identify protein binding partners of known proteins in an effort to discover new potential activities for a known protein.

A classical technique called “two-hybrid” has been used to identify the binding partner for a known protein that is described in, for example, Bartel and Fields (1997) The Yeast Two-Hybrid System, Oxford University Press New York. Although this technique has been successful in identifying single interacting partners for a given protein it is prone to error resulting in both false positives and false negatives and is limited in so far as the system is unable to identify higher order structures which are sometimes found within a cell.

A system to identify and isolate protein complexes is described in WO00/09716. The technique is referred to as tandem affinity purification (TAP) and makes use of a two part affinity purification method which utilises a fusion protein containing two different affinity tags, e.g. A and B, separated by a cleavable linker. Typically, a nucleic acid encoding a target protein is sub-cloned adjacent to one of the affinity tags. This creates a fusion protein that consists of:

NH target protein: affinity tag B-cleavable linker-affinity tag A COOH.

The fusion construct is transfected into a cell, for example a yeast cell, and is expressed. Proteins which bind the target become associated with the fusion protein and the cells are broken under non-denaturing conditions and the cell extract is applied to an affinity matrix to which tag A binds. The bound complex is then dis-associated from the affinity matrix after washing by cleavage of the linker, typically a protease sensitive linker. A second round of affinity purification is then conducted with a second affinity matrix to which affinity tag B binds. The second selection step is washed and eluted from the second matrix to provide a purified complex of proteins that is bound to the target protein. The two-step selection reduces non-specific binding and allows the isolation of a complex of proteins as opposed to a single binding partner. In WO00/09716 the first and second affinity tags are Protein A and cahnodulin binding protein. A further example of TAP is described in WO03/095619. In this example the first and second affinity tags is a protein with a biotinylation recognition motif and a hexapeptide His tag polypeptide respectively.

There is a desire to identify further affinity tags which have increased affinity for an affinity matrix to further reduce background binding by increasing the binding affinity of an affinity tag for its binding partner on the affinity matrix.

The colicins are a family of protein antibiotics that are made by the Enterobacteriacae during times of stress. These proteins kill susceptible bacterial cells either by acting as ionophores and depolarising the inner membrane or by lytic (e.g. nuclease) activity in the periplasm or cytoplasm. Typically, a colicin comprises a central receptor domain, an amino-terminal translocation domain and a carboxyl-terminal cytotoxic domain. A class of colicins, which are referred to as the enzymatic E class colicins, gains entry into a bacterial cell via contact with the vitamin B₁₂ receptor and the Tol complex located in the periplasm which triggers translocation of the colicin into the cell. Nine group E colicins are known; E1 is an ionophore forming colicin, the remainder are either endonucleases or ribonucleases. For example the E3, E4, E5 and E6 colicins are ribonucleases and the E2, E7, E8 and E9 are non specific DNases.

The expression of an E type colicin in a host bacterial cell is problematic since the host cell has to be able to tightly control the activity of the nuclease otherwise the host cell nucleic acid becomes sensitive to nuclease attack. This is overcome by the co-expression of so called “immunity proteins” which are inhibitors of colicins that bind the colicin nuclease domain to neutralise its activity. The complex of immunity protein and colicin is secreted into the extracellular environment and it is this complex that binds a target cell. Once translocation is initiated the immunity protein disassociates, leaving the target cell unprotected against the action of the colicin. An example of such an immunity protein is Im9 that binds with extremely high affinity to the endonuclease (DNase) domain of colicin E9 where, the K_{d} of the complex is 10⁻¹⁶M at pH 7 and 25° C. (Wallis et al (1995) Biochemistry 34, 13743). Another example is the immunity protein Im3 that binds to the ribonuclease (RNase) domain of colicin E3, where the K_{d} of the complex is 10⁻¹²M at pH 7 and 25° C. (Walker et al (2003) Biochemistry 42, 4161). These very high affinities make nuclease-immunity protein complexes as ideal affinity-based purification modules.

A further attraction in the use of colicin DNase-Im protein complexes in affinity-based purifications is the similarly high levels of specificity exhibited by the DNase-domains for their cognate in protein partners wherein non-cognate Im proteins are highly discriminated against (Li et al (2004) J. Mol. Biol. 337, 743). For example, the Im7 protein, specific for the DNase domain of colicin E7, binds to the non-cognate E9 DNase domain with a K_{d} of ˜10⁻⁴M, 12-orders of magnitude weaker than the cognate immunity protein Im9 under the same conditions. Therefore, and as described in the following, double-immunity protein fusions can be constructed in which the binding to colicin nuclease columns is specific to the cognate partnership.

We herein disclose an affinity purification methodology that exploits the very high affinity and specificity of immunity proteins for colicin nucleases.

According to an aspect of the invention there is provided a chimeric fusion protein comprising an immunity polypeptide linked to at least one heterologous polypeptide.

Preferably said immunity polypeptide is linked by a linker molecule to said heterologous polypeptide.

In a preferred embodiment of the invention said linker comprises a cleavable peptidic linker.

According to an aspect of the invention there is provided a nucleic acid molecule which encodes a chimeric polypeptide which nucleic acid molecule comprises:

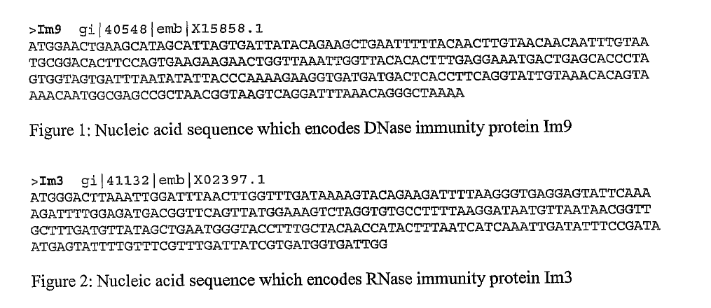

i) a first part consisting of a nucleic acid sequence as represented in FIG. 1 or 2 and which encodes at least one polypeptide, or active binding part thereof, which has the activity associated with an immunity protein; or a variant nucleic acid molecule which hybridises to the nucleic acid molecule as represented in FIGS. 1 and 2 which encodes a polypeptide which has the activity associated with an immunity polypeptide; and
ii) a second part consisting of a nucleic acid sequence which encodes a heterologous polypeptide wherein said first and second parts are linked.

In a preferred embodiment of the invention said first and second nucleic acid molecules are linked by a linker molecule. Preferably said linker encodes a cleavable peptidic linker.

In a further preferred embodiment of the invention said hybridisation conditions are stringent conditions.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Allows Sequences that Share at Least 80% Identity to Hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Allows Sequences that Share at Least 50% Identity to Hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

In a preferred embodiment of the invention said first part comprises a nucleic acid molecule consisting of a nucleic acid sequence which encodes at least one colicin DNase immunity polypeptide.

In a preferred embodiment of the invention said immunity polypeptide is encoded by the nucleic acid sequence as shown in Table 1A or the amino acid sequence as shown in Table 1B.

Preferably said immunity polypeptide is selected from the group consisting of: Im2, Im7, Im8 and Im9.

In an alternative preferred embodiment of the invention said first part comprises a nucleic acid molecule consisting of a nucleic acid sequence which encodes at least one colicin RNase immunity polypeptide.

In a preferred embodiment of the invention said immunity polypeptide is encoded by the nucleic acid sequence as shown in Table 2A or the amino acid sequence as shown in Table 2B.

Preferably said immunity polypeptide is selected from the group consisting of: Im3, Im4, Im5 and Im6

In a further preferred embodiment of the invention said nucleic acid molecule encodes a chimeric polypeptide comprising at least two immunity polypeptides wherein said polypeptides are in-frame translational fusions.

In a preferred embodiment of the invention said nucleic acid molecule encodes two immunity polypeptides that bind a similar colicin polypeptide.

In an alternative embodiment of the invention said nucleic acid molecule encodes two immunity polypeptides that bind dissimilar colicin polypeptides.

In a preferred embodiment of the invention said cleavable linker comprises at least one protease sensitive site. Preferably said site is a cleavage site for a tobacco etch virus protease.

According to a further aspect of the invention there is provided a chimeric polypeptide encoded by a nucleic acid molecule according to the invention.

In a preferred embodiment of the invention said chimeric polypeptide comprises at least one part that comprises a variant amino acid sequence.

A variant polypeptide may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies.

In a preferred embodiment of the invention said chimeric polypeptide sequence has 40% or greater sequence identity with the polypeptides hereindisclosed and which retain the biological activity associated with said polypeptides, for example colicin nuclease activity or immunity protein activity.

The invention features parts of said chimeric polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences disclosed herein and which retain the requisite biological activity.

According to a further aspect of the invention there is provided a composition comprising a nucleic acid molecule or polypeptide according to the invention.

According to a further aspect of the invention there is provided a vector comprising a nucleic acid molecule according to the invention.

In a preferred embodiment of the invention said nucleic acid molecule is operably linked to a promoter that controls the expression of said chimeric polypeptide.

Preferably said nucleic acid molecule is adapted for eukaryotic expression. Typically said adaptation includes, by example and not by way of limitation, the provision of transcription control sequences (promoter sequences) which mediate cell/tissue specific expression. These promoter sequences may be cell/tissue specific, inducible or constitutive.

“Promoter” is an art recognised term and, for the sake of clarity, includes the following features which are provided by example only. Enhancer elements are cis acting nucleic acid sequences often found 5′ to the transcription initiation site of a gene (enhancers can also be found 3′ to a gene sequence or even located in intronic sequences). Enhancers function to increase the rate of transcription of the gene to which the enhancer is linked. Enhancer activity is responsive to trans acting transcription factors that have been shown to bind specifically to enhancer elements. The binding/activity of transcription factors (please see Eukaryotic Transcription Factors, by David S Latchman, Academic Press Ltd, San Diego) is responsive to a number of physiological/environmental cues that include, by example and not by way of limitation, intermediary metabolites (e.g. glucose), environmental effectors (e.g. heat).

Promoter elements also include so called TATA box and RNA polymerase initiation selection sequences that function to select a site of transcription initiation. These sequences also bind polypeptides that function, inter alia, to facilitate transcription initiation selection by RNA polymerase.

Adaptations also include the provision of selectable markers and autonomous replication sequences that facilitate the maintenance of said vector in either the eukaryotic cell or prokaryotic host. Vectors that are maintained autonomously are referred to as episomal vectors.

Adaptations which facilitate the expression of vector encoded genes include the provision of transcription termination/polyadenylation sequences. This also includes the provision of internal ribosome entry sites (IRES) that function to maximise expression of vector encoded genes arranged in bi-cistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

Vectors can be viral based and may be derived from viruses including adenovirus, retrovirus, adeno-associated virus, herpesvirus, lentivirus; vaccinia virus and baculovirus.

According to a further aspect of the invention there is provided a cell transfected with a nucleic acid or vector according to the invention.

In a preferred embodiment of the invention said cell is a eukaryotic cell.

Preferably said eukaryotic cell is a mammalian cell.

In an alternative preferred embodiment of the invention said cell is a plant cell.

In a still further preferred embodiment of the invention said cell is a yeast cell.

In an alternative preferred embodiment of the invention said cell is a prokaryotic cell.

According to a further aspect of the invention there is provided the use of the chimeric polypeptide according to the invention for the isolation of a complex of biological molecules.

In a preferred embodiment of the invention said complex of biological molecules comprises a complex comprising at least one protein. Preferably said complex is a complex of protein molecules.

According to a further aspect of the invention there is provided a method to isolate a complex of biological molecules from a plurality of biological molecules comprising providing a preparation comprising a plurality of biological molecules and at least one chimeric polypeptide according to the invention and incubating the preparation under conditions which allow the association of biological molecules in said preparation with said chimeric polypeptide and isolating the complex of biological molecules associated with said chimeric polypeptide.

According to a further aspect of the invention there is provided a method to isolate a complex of biological molecules from a plurality of biological molecules comprising the steps of:

i) providing a preparation comprising a plurality of biological molecules and at least one chimeric polypeptide according to the invention and incubating the preparation under conditions which allow the association of biological molecules in said preparation with said chimeric polypeptide;
ii) contacting the mixture with a first affinity matrix comprising a binding partner for said chimeric polypeptide to allow the binding of at least part of said chimeric polypeptide to said matrix and washing said matrix to remove biological molecules non-specifically bound to said chimeric polypeptide and said matrix;
iii) eluting from said matrix the bound chimeric polypeptide and associated biological molecules;
iv) contacting said eluted chimeric polypeptide with a second affinity matrix comprising a second binding partner for said chimeric polypeptide to allow binding of a different part of said chimeric polypeptide to said second affinity matrix; and
v) eluting said chimeric polypeptide from said second matrix and optionally releasing said biological molecules associated with said chimeric polypeptide.

It will be apparent to the skilled artisan that elution of the chimeric polypeptide and associated biological molecules may be achieved by methods well known in the art and include, by example, elution by alteration in pH, ionic conditions or by incubation with an agent, for example a chemical agent or a protease, which cleaves the bound chimeric polypeptide from the matrix.

In a preferred method of the invention said preparation comprises a cell adapted to express the chimeric polypeptide according to the invention, said cell providing the plurality of biological molecules.

In a preferred method of the invention said complex comprises at least one protein molecule. Preferably said complex comprises a complex of protein molecules.

It will be apparent that the method according to the invention may be adapted to isolate complexes of biological molecules from mixtures. These complexes may be protein complexes or, for example, a mixture of protein and nucleic acid e.g. chromatin. The method may also be used to isolate organelles or even whole cells or cell membranes from complex mixtures. The complexes may also be formed from in vitro transcription and/or translation assays formed from cell extracts.

In a preferred method of the invention said first affinity matrix comprises a colicin polypeptide that binds its cognate immunity polypeptide.

In a further preferred method of the invention said second affinity matrix comprises a colicin polypeptide different from the colicin polypeptide of the first affinity matrix.

In a preferred method of the invention said elution is obtained by incubation with a protease which cleaves said linker to release said chimeric polypeptide bound to said first matrix.

In a further preferred method of the invention said chimeric polypeptide includes a second protease sensitive site, cleavage of which releases said chimeric polypeptide from said second affinity matrix.

In a further preferred method of the invention said colicin is selected from the group consisting of: E2, E7, E8 and E9.

In an alternative preferred method of the invention said colicin is selected from the group consisting of: E3, E4, E5 and E6.

According to a further aspect of the invention there is provided an affinity matrix comprising a substrate and associated crosslinked or conjugated thereto at least one polypeptide encoded by a nucleic acid molecule comprising a nucleic acid sequence selected from the group consisting of:

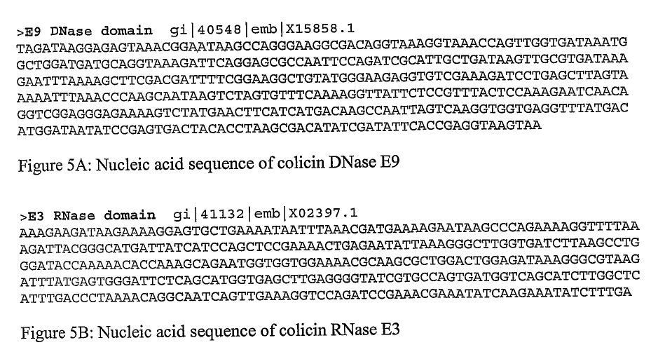

i) a nucleic acid molecule consisting of a nucleic acid sequence as represented in FIG. 5A or 5B;
ii) a nucleic acid molecule consisting of a nucleic acid sequence which hybridises to the nucleic acid molecule in (i) and which encodes a polypeptide with nuclease activity;
iii) a nucleic acid molecule which is degenerate as a result of the genetic code to the sequences defined in (i) and (ii) above.

In a preferred embodiment of the invention said nuclease is a colicin DNase.

In a preferred embodiment of the invention said colicin DNase is selected from the group consisting of: E2, E7, E8 and E9.

In an alternative preferred embodiment of the invention said nuclease is an RNase.

In a preferred embodiment of the invention said RNase is selected from the group consisting of: E3, E4, E5 and E6.

According to a further aspect of the invention there is provided a method for the coupling of at least one colicin polypeptide to a substrate comprising the steps of:

i) providing a preparation comprising a colicin polypeptide and a matrix material; and
ii) providing conditions which enable the association, cross-linking or conjugation of said colicin to said substrate.

In a preferred method of the invention said substrate is an affinity matrix material.

According to a further aspect of the invention there is provided a kit comprising: a nucleic acid or vector according to the invention or a polypeptide according to the invention; an agent which cleaves a cleavable linker in said chimeric polypeptide according to the invention; and affinity matrix materials required to isolate said chimeric polypeptide.

According to an aspect of the invention there is provided a chimeric fusion protein comprising a colicin polypeptide linked to at least one heterologous polypeptide.

According to a further aspect of the invention there is provided a nucleic acid molecule which encodes a chimeric polypeptide which nucleic acid molecule comprises:

i) a first part consisting of a nucleic acid sequence as represented in FIG. 5A or 5B and which encodes at least one polypeptide, or part thereof, which has nuclease activity; or a variant nucleic acid molecule which hybridises to the nucleic acid molecule as represented in FIG. 5A or 5B and which encodes a polypeptide which has nuclease activity; and
ii) a second part consisting of a nucleic acid sequence which encodes a heterologous polypeptide wherein said first and second parts are linked.

In a preferred embodiment of the invention said chimeric polypeptide is a variant polypeptide which has reduced nuclease activity. Preferably said chimeric polypeptide is a variant which lacks nuclease activity.

Aspects, embodiments, uses and methods applicable to chimeric polypeptides comprising immunity polypeptides are equally applicable to colicin comprising chimeric polypeptides.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1 is the nucleic acid sequence which encodes DNase immunity protein Im9;

FIG. 2 is the nucleic acid sequence which encodes RNase immunity protein Im3;

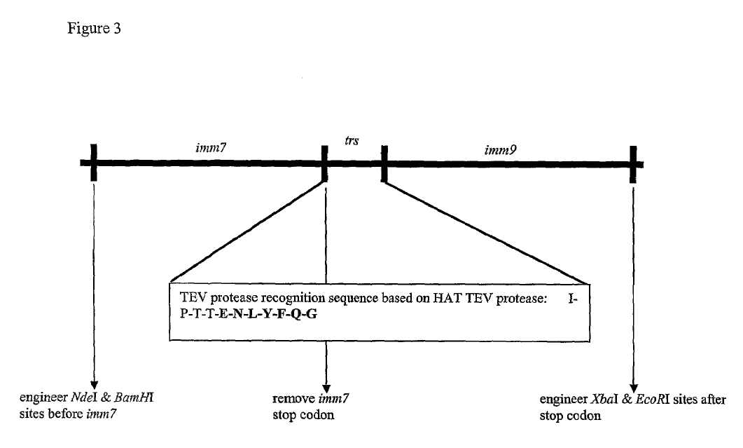

FIG. 3 is a schematic for double immunity protein tag showing the gene construction for dImP79, where Im7 and Im9 have been fused and are separated by a cleavage linker. trs: TEV protease recognition sequence based on highly aphid transmissable TEV protease;

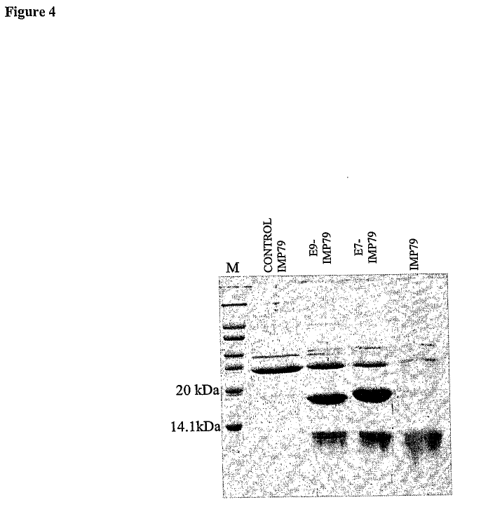

FIG. 4 shows a 16% SDS-PAGE of TEV protease cleavage of dImp79 in the presence of colicin DNases (E9 and E7) and alone (last lane). & Imp79 and Imp79 alone were subject to TEV protease cleavage;

FIG. 5A is the nucleic acid sequence of a colicin DNase domain E9; FIG. 5B is the nucleic acid sequence of a colicin RNase domain E3;

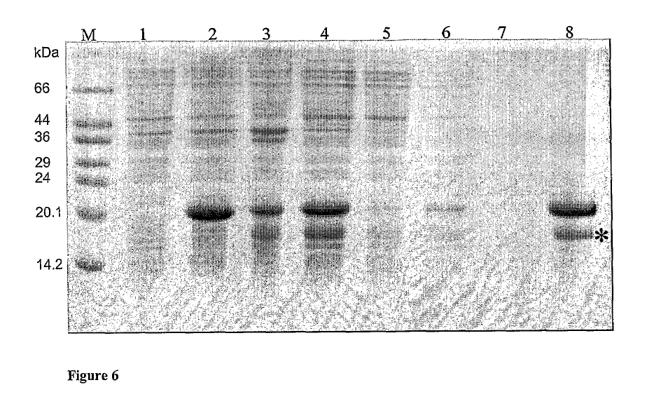

FIG. 6 shows a 16% SDS-PAGE of the purification steps using Im9 as a fusion purification tag. 1: uninduced cells of pNC3/BL21 DE3; 2: induced, overexpressed Im9 fused to a 34 residue peptide sequence (Im9Gol); 3&4: pellet and supernatant respectively, after cell disruption. Supernatant (lane 4) was loaded onto an E9 DNase-linked sepharose 4B column; 5: flow through off column during loading; 6&7: high salt washes of column; 8: eluted and dialysed protein Im9Gol. *, denotes a breakdown product of the fusion protein;

Table 1A (amino acid) and B (DNA) illustrates an alignment from the immunity proteins of the DNase family, based on the Im9 protein showing greater than 40% sequence identity. Representative proteins are shown for each sub-family such that OrfU1 represents the family of OrfU1 proteins that share at least 94% sequence identity. Programmes used were BLAST (at http://www.ncbi.nlm.nih.gov/BLAST) and ClustalW (at http://n-psa-pbil.ibcp.fr/cgi-bin/align_clustalw.pl),

Table 2A (amino acid) and B (DNA) illustrates proteins from the RNase immunity family as based on the Im3 protein showing greater than 40% sequence identity. Also included within this family is the protein from Pseudomonas fluorescens (alignment not shown). (http://npsa-pbil.ibcp.fr/cgi-bin/align_clustalw.pl):

Table 3A (amino acid) and B (DNA) illustrates alignment of the DNase family, based on the E9 DNase protein showing greater than 40% sequence identity. Representative proteins are shown for each sub-family such that Uro_Ecoli represents the family of uropathogenic specific proteins that share at least 78% sequence identity. Programmes used were BLAST (at http://www.ncbi.nhn.nih.gov/BLAST) and ClustalW (at http://npsa-pbil.ibgp.fr/cgi-bin/align_clustalw.pl); and

Table 4A (amino acid) and B (DNA) illustrates an alignment of RNases based on homology to E3 RNase where proteins are identified by greater than 40% homology. Representative sequences are shown for each protein family such that the protein from Pseudomonas fluorescens is typical of the family. Programmes used were BLAST (at http://www.ncbi.nlm.nih.gov/BLAST) and ClustalW (at http://npsa-pbil.ibcp.fr/cgi-bin/align_clustalw.nl).

### Materials and Methods

### Construction of Double Immunity Genes and Variants

The plasmids pRJ347 & pRJ345 (coding imm7 & imm9 genes respectively) were used as the templates. The 5′ gene of the double tag was engineered to include an NdeI and a BamHI restriction site in frame to the gene. The 3′ gene was designed to include XbaI and EcoRI restriciton sites after the stop codon. Primers were designed to amplify the genes, including overhang to code for the central TEV protease recognition sequence based on HAT TEV protease. The 5′ gene in the construct had the stop codon removed, allowing for direct readthrough. The two first round per products were used in a further per round where both products were used to anneal to each other. The final per product was purified and cloned into the Zero Blunt TOPO PCR Cloning Kit (Invitrogen) and used to transform E. coli TOP10 competent cells (Invitrogen). PCR colony screens identified gene fragments of the correct length. These colonies were amplified and the plasmids purified (Qiagen QIAprep Spin Miniprep Kit). DNA sequencing verified the sequence. To further weaken any possible interactions of the 2^{nd} tag to the E9 DNase resin, the Im7(D52A, Im7 numbering) variant of the tags was constructed using whole plasmid site directed mutagenesis, according to manufacturers instructions (Stratagene, Pfu Turbo DNA polymerase). Genes constructed in this way include dImP97 (imm9-trs-imm7), dImP97a, dImp79 and dImP7a9. A schematic of the gene construction is shown below.

Purification and Assay of the dImP Tag

dImP tags were assayed for in vitro viability by excising the genes from the cloning vector with NdeI, EcoRI, ligating into similarly restricted pET22b (Novagen) and transforming competent E. coli BL21 DE3. Vector with insert was identified by colony pcr screen and these plasmids named pIMP79, pIMP7a9, pIMP97 and pIMP97a. Trials were conducted for protein expression prior to protein purification. dImP protein was produced in E. coli growing in LB-amp (100 μg/ml) at 37° C. and induced with 1 mM IPTG during exponential growth. Cells were harvested, sonicated and purified essentially as for Immunity proteins (Wallis et al., 1992). Purified proteins were dialysed into 20 mM TEA, pH 7.5, snap frozen and stored at −20° C.

TEV Protease Cleavage of dImP in the Presence and Absence of Cognate DNase Domains

Protease cleavage was carried out for E9-Imp79, E7-Imp79 & Imp79 to assess cleavage of the dImP in complex with the cognate DNases. Approximately 40 μg of Imp79 (in complex with E7 DNase, E9 DNase domain, or alone) was incubated with 20 U of TEV protease for 1 hr 40 mins at 30° C. in buffer (50 mM Tris-HCl, pH7.5, 100 mM NaCl).

### Cross Linking E-DNase Domains to Sepharose

Purified E9 and E7 DNase domains (Garinot-Schneider et al., 1996) were cross linked to CNBr-activated Sepharose 4B beads (Amersham Biosciences) according to manufacturers guidelines. Im9 was also cross-linked to the resin in a similar manner.

### Complexes in the Presence of Detergents

As a test of complex formation in the presence of detergents, both the E9 DNase-Im9 and E3 RNase-Im3 complexes were preformed in detergent (β-octylglucoside upto 1% w/v; Triton X100 upto 1% v/v) and analysed for complex formation by size exclusion chromatography. Detergents do not destroy complexation of nuclease domains and their immunity proteins. These experiments show that the complexes survive the addition of detergents and will also form complexes when detergents are added prior to complex formation. The detergent NP40 was used (upto 0.5% v/v) in buffer for E9 DNase cross-linked Sepharose and adding the dImP protein tag. The tag still binds to the resin in the presence of detergent.

### Immunity Proteins as Purification Fusion Tags for Polypeptides

The imm9 gene was engineered to extend its sequence to include a TEV protease recognition site C-terminal. The gene construct was designed to include restriction sites for the cloning of target proteins 3′ to the protease site. The test system was the Im9Gol fusion protein, where Gol is a 35 amino acid polypeptide co-activator in bacteriophage exclusion systems. Restriction sites are (5′-3′): NdeI(imm9)(trs)XhoIHindIII(gol)BamHI. The gene construct was ligated into pET11c and pET15b vectors (Novagen). Purification of overexpressed product is carried out using either anion exchange chromatography or E9 DNase-cross linked resin.

TABLE 1

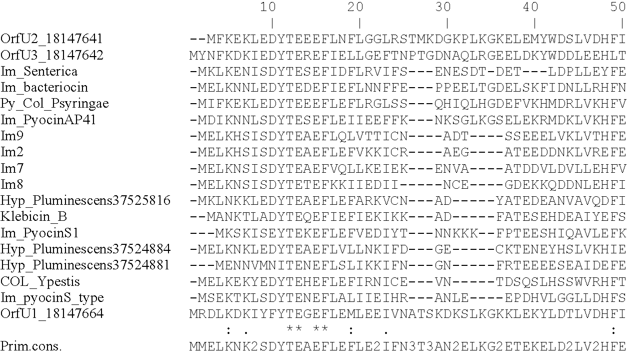

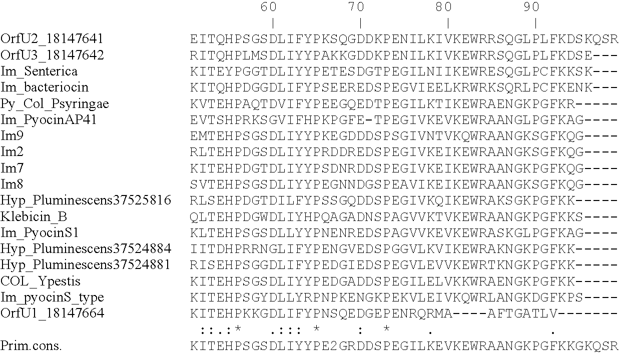

TABLE 2

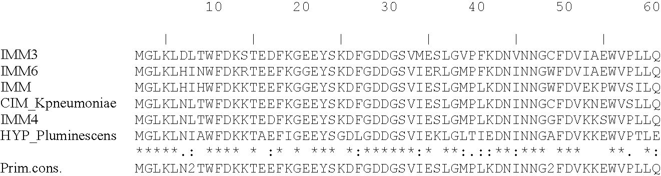

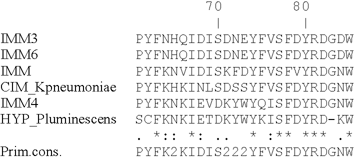

TABLE 3

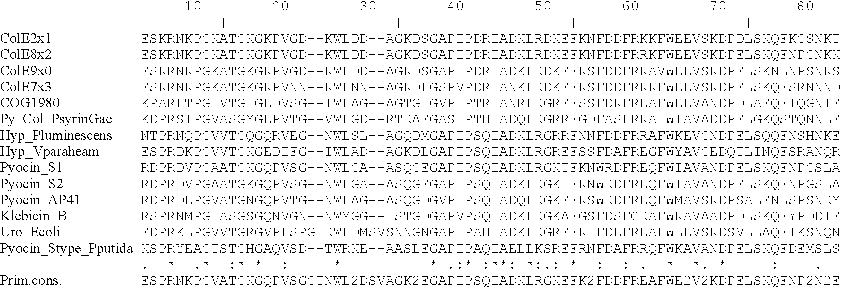

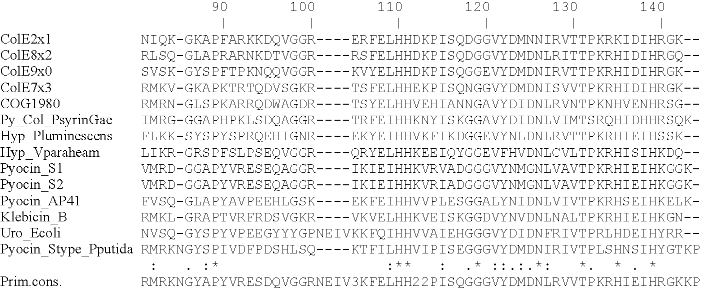

TABLE 4

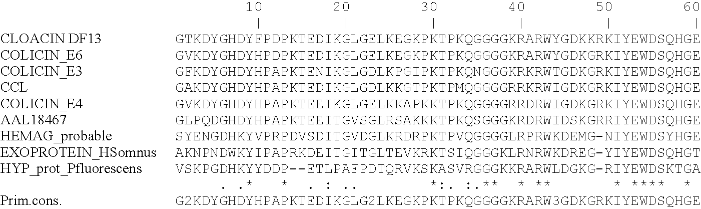

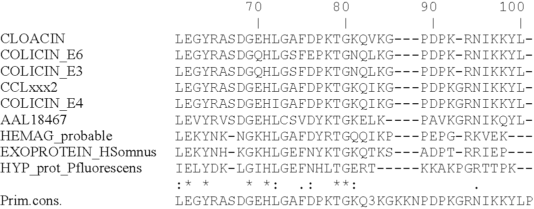

1. A chimeric fusion protein comprising an immunity polypeptide linked to at least one heterologous polypeptide.

2. A protein according to claim 1 wherein said immunity polypeptide is linked by a linker molecule to said heterologous polypeptide.

3. A protein according to claim 2 wherein said linker comprises a cleavable peptidic linker.

4. A nucleic acid molecule which encodes a chimeric polypeptide which nucleic acid molecule comprises:
i) a first part consisting of a nucleic acid sequence as represented in FIG. 1 or 2 and which encodes at least one polypeptide, or active binding part thereof, which has the activity associated with an immunity protein; or a variant nucleic acid molecule which hybridises to the nucleic acid molecule as represented in FIG. 1 or FIG. 2 and which encodes a polypeptide which has the activity associated with an immunity polypeptide; and
ii) a second part consisting of a nucleic acid sequence which encodes a heterologous polypeptide wherein said first and second parts are linked.

5. A nucleic acid molecule according to claim 4 wherein said first and second nucleic acid molecules are linked by a linker molecule.

6. A nucleic acid according to claim 5 wherein said linker encodes a cleavable peptidic linker.

7. A nucleic acid molecule according to claim 4 wherein said first part comprises a nucleic acid molecule consisting of a nucleic acid sequence which encodes at least one colicin DNase immunity polypeptide.

8. A nucleic acid molecule according to claim 7 wherein said immunity polypeptide is selected from the group consisting of: Im2, Im7, Im8 and Im9.

9. A nucleic acid molecule according to claim 4 wherein said first part comprises a nucleic acid molecule consisting of a nucleic acid sequence which encodes at least one colicin RNase immunity polypeptide.

10. A nucleic acid molecule according to claim 9 wherein said immunity polypeptide is selected from the group consisting of: Im3, Im4, Im5 and Im6.

11. A nucleic acid molecule according to claim 4 which encodes a chimeric polypeptide comprising at least two immunity polypeptides wherein said polypeptides are in frame translational fusions.

12. A nucleic acid molecule according to claim 11 wherein said nucleic acid molecule encodes two immunity polypeptides which bind a similar colicin polypeptide.

13. A nucleic acid molecule according to claim 11 wherein said nucleic acid molecule encodes two immunity polypeptides which bind dissimilar colicin polypeptides.

14. A nucleic acid molecule according to claim 6 wherein said cleavable linker comprises at least one protease sensitive site.

15. A nucleic acid according to claim 14 wherein said site is a cleavage site for a tobacco etch virus protease.

16. A chimeric polypeptide encoded by a nucleic acid molecule according to claim 4.

17. A composition comprising a nucleic acid molecule or polypeptide according to claim 1.

18. A vector comprising a nucleic acid molecule according to claim 4.

19. A cell transfected with a nucleic acid or vector according to claim 4.

20. A cell according to claim 19 wherein said cell is a eukaryotic cell.

21. A cell according to claim 20 wherein said eukaryotic cell is a mammalian cell.

22. A cell according to claim 20 wherein said cell is a plant cell.

23. A cell according to claim 20 wherein said cell is a yeast cell.

24. A cell according to claim 19 wherein said cell is a prokaryotic cell.

25. The use of the chimeric polypeptide according to claims 1 for the isolation of a complex of biological molecules.

26. Use according to claim 25 wherein said complex of biological molecules comprises a complex of at least one protein.

27. Use according to claim 26 wherein said complex is a complex of protein molecules.

28. A method, to isolate a complex of biological molecules from a plurality of biological molecules comprising providing a preparation comprising a plurality of biological molecules and at least one chimeric polypeptide according to claim 1 and incubating the preparation under conditions which allow the association of biological molecules in said preparation with said chimeric polypeptide and isolating the complex of biological molecules associated with said chimeric polypeptide.

29. A method to isolate a complex of biological molecules from a plurality of biological molecules comprising the steps of:
i) providing a preparation comprising a plurality of biological molecules and at least one chimeric polypeptide according to claim 1 and incubating the preparation under conditions which allow the association of biological molecules in said preparation with said chimeric polypeptide;
ii) contacting the mixture with a first affinity matrix comprising a binding partner for said chimeric polypeptide to allow the binding of at least part of said chimeric polypeptide to said matrix and washing said matrix to remove biological molecules non-specifically bound to said chimeric polypeptide and said matrix;
iii) eluting from said matrix the bound chimeric polypeptide and associated biological molecules;
iv) contacting said eluted chimeric polypeptide with a second affinity matrix comprising a second binding partner for said chimeric polypeptide to allow binding of a different part of said chimeric polypeptide to said second affinity matrix; and
v) eluting said chimeric polypeptide from said second matrix and optionally releasing said biological molecules associated with said chimeric polypeptide.

30. A method according to claim 29 wherein said preparation comprises a cell adapted to express said chimeric polypeptide.

31. A method according to claim 29 wherein said complex comprises at least one protein molecule.

32. A method according to claim 31 wherein said complex comprises a complex of protein molecules.

33. A method according to claim 29 wherein said first affinity matrix comprises a colicin polypeptide which binds its cognate immunity polypeptide.

34. A method according to claim 29 wherein said second affinity matrix comprises a colicin polypeptide different from the colicin polypeptide of the first affinity matrix.

35. A method according to claim 29 wherein said elution is obtained by incubation with a protease which cleaves said linker to release said chimeric polypeptide bound to said first matrix.

36. A method according to claim 29 wherein said chimeric polypeptide includes a second protease sensitive site, cleavage of which releases said chimeric polypeptide from said second affinity matrix.

37. A method according to claim 33 wherein said colicin polypeptide is selected from the group consisting of: E2, E7, E8 and E9.

38. A method according to claim 33 wherein said colicin polypeptide is selected from the group consisting of: E3, E4, E5 and E6.

39. An affinity matrix comprising a substrate and associated crosslinked or conjugated thereto at least one polypeptide encoded by a nucleic acid molecule comprising a nucleic acid sequence selected from the group consisting of:
i) a nucleic acid molecule consisting of a nucleic acid sequence as represented in FIG. 5A or 5B;
ii) a nucleic acid molecule consisting of a nucleic acid sequence which hybridises to the nucleic acid molecule in (i) and which encodes a polypeptide with nuclease activity;
iii) a nucleic acid molecule which is degenerate as a result of the genetic code to the sequences defined in (i) and (ii) above.

40. A matrix according to claim 39 wherein said nuclease is a colicin DNase polypeptide.

41. A matrix according to claim 40 wherein said colicin DNase polypeptide is selected from the group consisting of: E2, E7, E8 and E9.

42. A matrix according to claim 39 wherein said nuclease is an RNase polypeptide.

43. A matrix according to claim 42 wherein said RNase polypeptide is selected from the group consisting of: E3, E4, E5 and E6.

44. A method for the coupling of at least one colicin polypeptide to a substrate comprising the steps of:
ii) providing a preparation comprising a colicin polypeptide and a matrix material; and
iii) providing conditions which enable the association, cross-linking or conjugation of said colicin to said substrate.

45. A method according to claim 44 wherein said substrate is an affinity matrix material.

46. A kit comprising: a nucleic acid or vector according to claim 4, or a polypeptide according to claim 1; an agent which cleaves a cleavable linker in said chimeric polypeptide; and affinity matrix materials required to isolate said chimeric polypeptide.
